(19) United States
(12) Patent Application Publication
(10) Pub. No.: US 2021/0154424 A1
(43) Pub. Date: May 27, 2021

(54) IMPROVED TRACHEOSTOMY DEVICE

(71) Applicant: LANCASHIRE TEACHING HOSPITALS NHS FOUNDATION TRUST, Fulwood, Preston, Lancashire (GB)

(72) Inventors: Shondipon LAHA, Fulwood, Preston, Lancashire (GB); Alexander Chukwudera ADIELE, Bailrigg, Lancaster (GB); Damian BREWER, Bailrigg, Lancaster (GB); Theodoros NTELIS, Bailrigg, Lancaster (GB); Samantha PITMAN, Bailrigg, Lancaster (GB); Allan Edward Watson RENNIE, Bailrigg, Lancaster (GB); Yasar SHEIKH, Bailrigg, Lancaster (GB)

(21) Appl. No.: 16/610,785

(22) PCT Filed: May 3, 2018
(86) PCT No.: PCT/GB2018/051193

§ 371 Date: Nov. 4, 2019

(30) Foreign Application Priority Data
May 3, 2017 (GB) 1707060.8

(51) Int. Cl. A61M 16/04

(52) U.S. Cl. CPC A61M 16/0465; A61M 16/0427

(57) ABSTRACT

A tracheostomy airway device is provided comprising an outer cannula (110) having open proximal and distal ends, an inner actuation member insertable into the proximal end of the outer cannula, and one or more retention members (130). The one or more retention members are movable relative to the outer cannula between a retracted configuration in which the tracheostomy device is insertable into, and removable from, the trachea of a patient, and an extended configuration in which the one or more retention members impede removal of the tracheostomy airway device from the trachea. The retention members are operably linked to the inner actuation member or the outer cannula, such that movement of the inner actuation member relative to the outer cannula actuates movement of the one or more retention members between the retracted configuration and the extended configuration.

The present invention relates to an improved tracheostomy device.

The trachea is a conduit between the upper airway and the lungs that delivers moist warm air to the lungs, and returns carbon dioxide and sputum from the lungs. A tracheostomy is an operative procedure that consists of a surgical procedure to the anterior of the neck and making a direct airway through a surgical opening in the trachea. The resulting stoma, or tracheostomy, can serve independently as an airway (in the long-term) or, more usually, as a site for a tracheostomy device to be inserted, and the tracheostomy device then serves as an airway that allows a person to breathe without the use of the nose or mouth.

A tracheostomy may be performed to bypass upper airway blockages, e.g. caused by severe facial trauma, tumours of the head and neck (e.g., cancers, branchial cleft cysts), or obstructive sleep apnoea, for example. A tracheostomy may also be performed in patients who have a need for long-term mechanical ventilation.

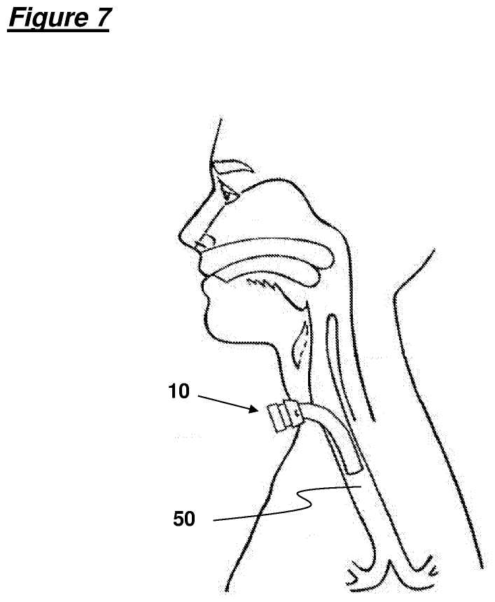

A typical tracheostomy device comprises a tube or cannula for insertion through an hole in the neck and trachea, such that a distal end of the cannula is located within the trachea. The tracheostomy device typically has one or more flanges at the proximal end of the tube for securing the tracheostomy device to the neck of the patient using ties, which are secured around the neck. A schematic view of a conventional tracheostomy airway device inserted into the trachea of a patient is provided in FIG. 7.

Traditionally, tracheostomy tubes are placed via an open technique in the operating room; however, the majority are now being performed in intensive care units via either open or percutaneous techniques. The complications that can arise in a tracheostomy include bleeding, accidental decannulation or tube dislodgement, mucous plugging, tracheoesophageal fistula, persistent tracheocutaneous fistula, tracheitis, tracheal stenosis and tracheoinnominate fistula.

Tracheostomy tube dislodgement can happen in any patient, but factors that increase the risk of dislodgment, a potentially catastrophic problem, include morbid obesity, a short or thick neck, a goiter, prior surgery of the neck, the device being connected to ventilator tubing, patient movement, frequent coughing and inadequately secured tubes. Complications of a dislodged tube include a loss of airway, a pneumothorax, subcutaneous emphysema, pseudotract formation, stomal stenosis, tracheoinnominate fistula and sternoclavicular osteomyelitis.

It is conventional, therefore, for clinicians to take great care securing tracheostomy devices to the neck of a patient in order to reduce the risk of tracheostomy tube dislodgement, and to be vigilant in respect of the causes of dislodgement and indications that dislodgement has occurred.

There has now been devised an improved tracheostomy device, which overcomes or substantially mitigates the aforementioned disadvantages associated with the prior art.

According to a first aspect of the invention, there is provided a tracheostomy airway device comprising an outer cannula having open proximal and distal ends, an inner actuation member insertable into the proximal end of the outer cannula, and one or more retention members, which are movable relative to the outer cannula between a retracted configuration in which the tracheostomy device is insertable into, and removable from, the trachea of a patient, and an extended configuration in which the one or more retention members impede removal of the tracheostomy airway device from the trachea, wherein the retention members are operably linked to the inner actuation member or the outer cannula, such that movement of the inner actuation member relative to the outer cannula actuates movement of the one or more retention members between the retracted configuration and the extended configuration.

The tracheostomy airway device according to the invention is advantageous principally because the provision of one or more moveable retention members may reduce the risk of dislodgement of the device, and hence may reduce the risk of complications arising from a dislodged tracheostomy airway device, including a loss of airway, a pneumothorax, subcutaneous emphysema, pseudotract formation, stomal stenosis, tracheoinnominate fistula and sternoclavicular osteomyelitis. Furthermore, the tracheostomy airway device according to the invention is advantageous because movement of the inner actuation member relative to the outer cannula actuates movement of the retention member between the retracted configuration and the extended configuration. This feature may enable a simple construction of the tracheostomy airway device, with reduced manufacturing costs, less risk of failure and less parts, relative to prior art retention mechanisms. This feature may also enable ready actuation of movement of the one or more retention members by a user, without the need for complex operating procedures.

The inner actuation member that is insertable into the proximal end of the outer cannula may have the form of an inner cannula. The inner actuation member may comprise a removable liner for facilitating cleaning of secretions that may accumulate, during use. The inner actuation member may have a form suitable for conveying respiratory gases between the ambient surroundings and/or a respiratory circuit and the interior of the outer cannula, and hence may comprise a gases conduit between the proximal and distal ends. The inner actuation member may be generally tubular in form, and may have substantially smooth exterior and/or interior surfaces, at least over a majority of its length, e.g. extending from the distal end of the inner actuation member.

The inner actuation member may have a regularly-shaped cross-section, which may be substantially uniform along its length, or at least a majority of its length. The inner actuation member may have a generally circular or elliptical cross-sectional shape. The exterior shape of the inner actuation member may substantially match the interior shape of the outer cannula, at least those portions that are brought alongside each other on insertion of the inner actuation member into the outer cannula, such that the inner actuation member is received within the outer cannula with a close fit.

Along its length, the inner actuation member may be generally arcuate in form, or may comprise a proximal portion that is generally linear in form, followed by an arcuate distal portion, such that the proximal and distal portions are formed at a non-zero angle, e.g. an oblique angle, relative to each other. In either arrangement, the device may comprise a proximal region that extends linearly, which is arranged to be received within a proximal region of the outer cannula that also extends linearly.

The inner actuation member may extend at least 10%, 20%, 30%, 40% or 50% along the length of the outer cannula, from the proximal end, in a fully inserted configuration and/or the extended configuration.

The inner actuation member may comprise a continuous wall, e.g. a tubular wall, or may comprise a discontinuous wall, e.g. having the form of one or more fingers extending from a base portion, e.g. at the proximal end of the inner actuation member. The proximal end of the inner actuation member may have a generally flat, annular end surface, which may be orientated generally perpendicularly relative to a central axis along which the outer cannula extends. The proximal end of the outer cannula may comprise an end portion having an enlarged external diameter, which may define an underside surface, which may be annular in form, that is suitable for abutment with a corresponding portion of the outer cannula when fully inserted, for example, in the extended configuration of the device.

In one embodiment, the inner actuation member has a tubular wall, which may be generally circular. The tubular wall may have a proximal portion with an enlarged external diameter, and hence an increased wall thickness, which defines a head of the inner actuation member. The external diameter of the inner actuation member head may be approximately equal to the internal diameter of the tubular wall of the outer cannula. The tubular wall may also comprise a cylindrical intermediate portion, extending along a linear central axis, and a distal portion that extends along an arcuate central axis, with the central axis of the distal portion having a substantially constant radius. The inner and outer diameters of the tubular wall may be uniform along the length of the inner cannula component, save for the inner actuation member head, for example, which may project radially outwardly from the tubular wall at the proximal end of the tubular wall. The outer diameter of the intermediate and distal portions of the inner actuation member may be approximately equal to the internal diameter of the tubular wall of the outer cannula. The inner actuation member head may be annular in shape, with flat annular abutment surfaces, and a cylindrical side surface. The inner and outer surfaces of the tubular wall, save for the inner actuation member head, for example, may be smooth, and the tubular wall may terminate at proximal and distal ends, which may comprise flat annular end surfaces, orientated perpendicularly to the adjacent central axis of the tubular wall.

The inner actuation member may be formed of plastics material(s), and the plastics material(s) may be sufficiently rigid for the inner actuation member to maintain its shape during use, and maintain the patency of a gases conduit defined therethrough. The inner actuation member may be formed of a material that is suitable for autoclaving, and hence suitable for re-use, or the inner actuation member may be formed of a material that is suitable for disposal following a single use. The inner actuation member may be a unitary component, in that the inner actuation member component may be formed as a single or combined (e.g. two-shot) moulding operation. The inner actuation member may be integrally formed with the one or more retention members, such that the inner actuation member and the one or more retention members are together formed as a unitary component. Alternatively, the inner actuation member may be mechanically connected or bonded to the one or more retention members, e.g. in an assembly step that follows moulding of the inner actuation member and the one or more retention members.

The proximal end of the inner actuation member may include a tubular connector, e.g. a 15 mm connector, for connecting a gases conduit of the inner actuation member and/or the outer cannula to respiratory apparatus, e.g. respiratory tubing.

The inner actuation member may be configured to be slidably received within the proximal end of the outer cannula. The inner actuation member may therefore be arranged for axial, sliding movement relative to the outer cannula. The inner actuation member may be rotationally fixed, or may be arranged for rotational movement, restricted or unrestricted.

The inner actuation member may comprise an engagement portion adapted to come into engagement, e.g. contact, with the one or more retention members. Alternatively, if the inner actuation member is fixed to the one or more retention members, the engagement portion may be adapted to come into engagement, e.g. contact, with the outer cannula or other engagement member fixed to the outer cannula.

The inner actuation member may be fixable relative to the outer cannula, e.g. following insertion and movement to the extended configuration, in order to provide a configuration suitable for long-term use of the device by the patient. The outer cannula and/or the inner actuation member may therefore comprise corresponding fastener formations, such as a snap-fit, bayonet or threaded connection.

The one or more retention members may be mounted relative to the outer cannula. In this arrangement, the one or more retention members may comprise an engagement portion that is engaged by a corresponding engagement portion of the inner actuation member, during insertion of the inner actuation member into the outer cannula, in use. The one or more retention members may be arranged for movement relative to the outer cannula as the inner actuation member is inserted into the outer cannula, and the engagement portion of the inner actuation member moves the engagement portion of the one or more retention members.

Alternatively, the one or more retention members may be mounted relative to the inner actuation member. In this arrangement, the one or more retention members may comprise an engagement portion that is engaged by a corresponding engagement portion of the outer cannula, during insertion of the inner actuation member into the outer cannula, in use. The one or more retention members may be arranged for movement relative to the outer cannula as the inner actuation member is inserted into the outer cannula, and the engagement portion of the outer cannula moves the engagement portion of the one or more retention members.

The engagement between the respective engagement portions may be a mechanical engagement, and the engagement portions may be adapted to cause an outward, e.g. radial, force to be imparted on the one or more retention members, e.g. from a linear force being applied to the inner actuation member. For example, at least one of these respective engagement portions may have an engagement surface that is orientated at an oblique angle relative to the direction of relative movement between the inner actuation member and the outer cannula. In addition, or alternatively, the one or more retention members may be mounted for rotational and/or pivoting movement relative to the outer cannula and/or the inner actuation member.

The one or more retention members may comprise a retaining portion, which projects outwardly, e.g. radially, relative to the exterior surface of the outer cannula in the extended configuration. The retaining portion may be arranged to impinge on the interior surface of the trachea of a patient, in the extended configuration of the device, in the event that a removal or dislodgement force and movement is applied to the device.

The one or more retention members may be connected to the outer cannula or the inner actuation member by a mechanical connection, e.g. a hinge. Alternatively, the one or more retention members may be mounted to the outer cannula or the inner actuation member by means of a fixed portion of the one or more retention members, which does not move relative to the component to which it is fixed, and the one or more retention members being deformable. The portion of the one or more retention members that is deformable relative to the fixed portion may comprise the retaining portion and/or the engagement portion of the one or more retention members. The one or more retention members may be mounted to the outer cannula or the inner actuation member by means of fastening, bonding, adhesive, welding, co-moulding or any other suitably robust connection. The connection may be non-releasable, without breaking the connection with excessive force, such that the one or more retention members cannot become detached in use.

The one or more retention members may be subject to a biasing force, which acts to urge the one or more retention members to a rest position. The one or more retention members may therefore be urged by the biasing force to return to a rest position, which may correspond to the position of the one or more retention members in a retracted configuration, when the inner actuation member is withdrawn, e.g. at least partially or fully, from the outer cannula. The biasing force may be the effect of deformation of a resilient material, or may be caused by impingement of the one or more retention members on a component, such as the outer cannula, as the inner actuation member is withdrawn.

The one or more retention members may be disposed in a proximal region of the device. The one or more retention members may be separated from the proximal end of the outer cannula, but may be disposed closer to the proximal end of the outer cannula than the distal end of the outer cannula. The device may comprise a plurality of retention members, e.g. three or more retention members, which may be arranged regularly about a circumference of the exterior of the outer cannula, at least in the extended configuration.

The retaining portion of the one or more retention members may comprise an abutment surface arranged to impinge on an interior surface of the trachea of a patient, in the event of a withdrawal or dislodgement force or movement. The abutment surface may be substantially flat or arcuate in form. The abutment surface may face an interior surface of the trachea in the extended configuration, when inserted. The abutment surface may be orientated obliquely relative to the axis along which the corresponding portion of the outer cannula extends, and relative to the interior surface of the trachea. The retaining portion of the one or more retention members may be generally planar, or generally cylindrical, in form.

The retaining portion may be disposed alongside, e.g. to the exterior or radially-outward side, or within, an opening in the outer cannula in the retracted configuration. The retaining portion may be movable between a position in the retracted configuration where the abutment surface is substantially aligned with the exterior surface of the outer cannula, and a position in the extended configuration in which the retaining portion and/or the abutment surface projects outwardly relative to the exterior surface of the outer cannula, and may be orientated at an oblique or perpendicular angle thereto.

The engagement portion of the one or more retention members may be mounted to the interior side, e.g. radially-inward side, of the retaining portion. The engagement portion may be disposed alongside, e.g. to the interior or radially-inward side, or within, an opening in the outer cannula in the retracted configuration. The engagement portion may be movable between a position in the retracted configuration where the engagement portion and/or engagement surface is at least partially disposed within the interior of the outer cannula, and a position in the extended configuration in which the engagement portion and/or engagement surface is at least partially removed the interior of the outer cannula. Hence, the engagement portion may project into the interior of the outer cannula to a lesser extent in the extended configuration than in the extended configuration.

The retracted configuration may enable insertion of the outer cannula through an incision or puncture of a patient's neck and removal therefrom. In this configuration, the retaining portion may either not project relative to the exterior surface of the outer cannula, or may project a distance less than 20%, 10% or 5% of the diameter of the outer cannula.

The extended configuration may enable the one or more retention members to impede removal of the tracheostomy airway device from the trachea. In particular, the one or more retention members may extend outwardly from the exterior surface of the outer cannula to a greater extent than in the retracted configuration. In the extended configuration, the retaining portion may project a distance greater than 5%, 10%, 20%, 30%, 40%, 60% or 80% of the diameter of the outer cannula.

The one or more retention members are operably linked to the inner actuation member or the outer cannula, such that movement of the inner actuation member relative to the outer cannula actuates movement of the one or more retention members between the retracted configuration and the extended configuration. The inner actuation member may be insertable to a passive insertion position without causing movement, or at least significant movement, of the one or more retention members, such that the device remains in a retracted configuration. The inner actuation member may be moveable to an active insertion position, which insertion causes movement of the one or more retention members between the retracted configuration and the extended configuration. The movement to the active insertion position, from the passive insertion position, may be axial, e.g. linear, movement and/or non-axial, e.g. rotational, movement.

The outer cannula may have a form suitable for conveying respiratory gases between the ambient surroundings and/or a respiratory circuit and the trachea of the patient, and hence may comprise a gases conduit between the proximal and distal ends. The outer cannula may be generally tubular in form, and may have substantially smooth exterior and/or interior surfaces, at least over a majority of its length, e.g. extending from the distal end of the device.

The outer cannula may have a regularly-shaped cross-section, which may be substantially uniform along its length, or at least a majority of its length. The outer cannula may have a generally circular or elliptical cross-sectional shape. Along its length, the outer cannula may be generally arcuate in form, or may comprise proximal and distal portions that are generally linear in form, connected by an arcuate intermediate portion, such that the proximal and distal portions are formed at a non-zero angle, e.g. an oblique angle, relative to each other. In either arrangement, the device may comprise a proximal region that extends linearly, which is arranged to receive the actuation member.

The outer cannula may also comprise an outwardly, e.g. radially, extending abutment flange, which may extend from the exterior of the outer cannula near to, but separated from, the proximal end of the outer cannula. The abutment flange may be adapted to contact, e.g. abut, or lie alongside the exterior of a patient's neck, in use, in order to facilitate retention of the device. The abutment flange may include outwardly extending wings, e.g. diametrically-opposed wings, which may include or be connectable to fasteners, e.g. by means of corresponding openings, for fixing the device to a patient's neck. The fasteners may be flexible members that may be tied around a patient's neck

The outer cannula may have a generally circular tubular wall, which may have a short proximal portion that is generally cylindrical in shape, and the outer cannula may have a main portion that extends along an arcuate central axis, and the central axis may have a substantially constant radius. The inner and outer diameters of the tubular wall may be substantially uniform along the length of the outer cannula, but the outer cannula may have an abutment flange, which may project radially outwardly from the tubular wall near to, but separately slightly from, a proximal end of the tubular wall. The abutment flange may separate a generally cylindrical proximal portion of the tubular wall and a main portion of the tubular wall that extends along an arcuate central axis. The abutment flange may be annular in shape, with flat annular abutment surfaces, and a cylindrical side surface. The inner and outer surfaces of the tubular wall, save for the abutment flange, may be smooth, and terminate at proximal and distal ends, which may comprise flat annular end surfaces, e.g. orientated perpendicularly to the adjacent central axis of the tubular wall.

The outer cannula may be formed of plastics material, and the plastics material may be sufficiently rigid for the outer cannula to maintain its shape during use, and maintain the patency of the gases conduit defined therethrough. The outer cannula may be formed of a material that is suitable for autoclaving, and hence suitable for re-use, or the outer cannula may be formed of a material that is suitable for disposal following a single use. The outer cannula may be a unitary component, in that the outer cannula component may be formed as a single or combined (e.g. two-shot) moulding operation. The outer cannula may be integrally formed with the one or more retention members, such that the outer cannula and the one or more retention members are together formed as a unitary component. Alternatively, the outer cannula may be mechanically connected or bonded to the one or more retention members, e.g. in an assembly step that follows moulding of the outer cannula and the one or more retention members.

The proximal end of the outer cannula may have a generally flat, annular end surface, which may be orientated generally perpendicularly relative to a central axis along which the outer cannula extends. The proximal end of the outer cannula may therefore be suitable for abutment with a corresponding portion of the actuation member when fully inserted, for example, in the extended configuration of the device.

The proximal end of the outer cannula may include a tubular connector, e.g. a 15 mm connector, for connecting the gases conduit of the outer cannula to respiratory apparatus, e.g. respiratory tubing. Alternatively, a tubular connector may be provided at the proximal end of the actuation member for this purpose.

The tracheostomy device may also be provided with a placement guide or obturator, which may be elongated, may be formable, and may include a cushioned tip.

The tracheostomy device may include a cuff, in a distal region of the exterior surface of the outer cannula, for sealing against the interior surface of the trachea. The cuff may be inflatable, and hence the tracheostomy device may comprise an inflation line, which may be fixed to the exterior surface of the outer cannula, and may comprise a pilot balloon for effecting inflation and deflation of the cuff, in use. The cuff may extend circumferentially around the exterior of the outer cannula, and be disposed further towards the distal end of the outer cannula relative to the one or more retention members.

The tracheostomy device may be adapted for human or animal use, for adults or children. The tracheostomy device may have an overall length of between 50 mm and 150 mm, or between 50 mm and 100 mm.

The tracheostomy device may include fenestration openings, which may be formed in the wall of the outer cannula. The tracheostomy device may also include a plug for the proximal end of the outer cannula or the inner actuation member.

According to a further aspect of the invention, there is provided a method of inserting a tracheostomy airway device, such as the tracheostomy device defined above, comprising:

(a) providing the tracheostomy device in a retracted configuration in which the tracheostomy device is insertable into the trachea of a patient;

(b) inserting an outer cannula of the tracheostomy device into the trachea of a patient;

(c) inserting an inner actuation member of the tracheostomy device into a proximal end of the outer cannula; and

(d) moving the inner actuation member relative to the outer cannula, thereby actuating movement of one or more retention members of the tracheostomy device into an extended configuration in which the one or more retention members impede removal of the tracheostomy airway device from the trachea.

The method according to the invention may include the further step, for decannulation, of moving the inner actuation member relative to the outer cannula, thereby actuating movement of one or more retention members of the tracheostomy device into the retracted configuration in which the tracheostomy device is removable from the trachea of a patient.

A preferred embodiment of the invention will now be described, by way of illustration only, with reference to the accompanying drawings, in which:

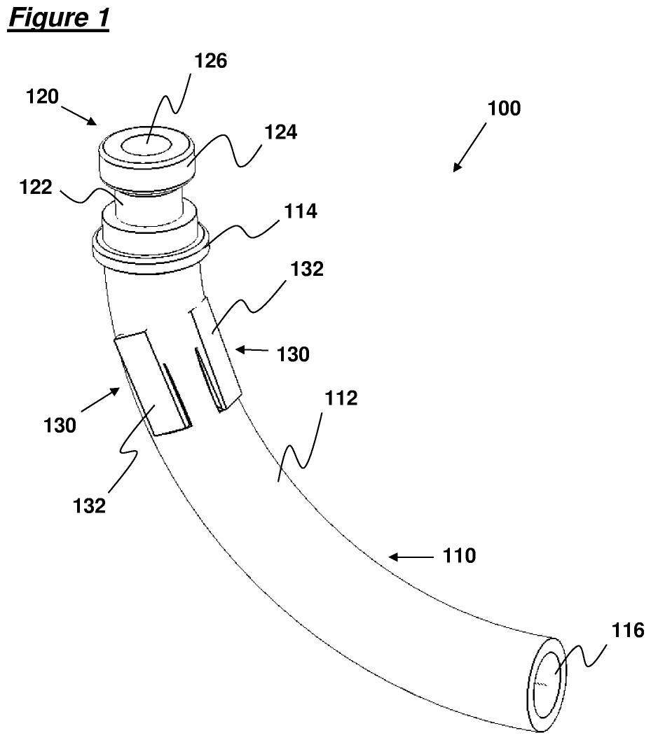

FIG. 1 is a first perspective view of a tracheostomy airway device according to the invention, in a retracted configuration;

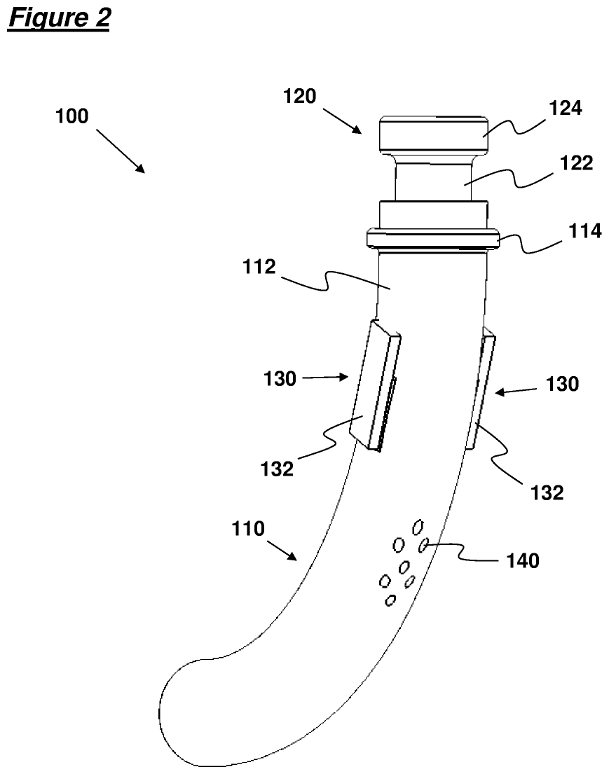

FIG. 2 is a second perspective view of the tracheostomy airway device of FIG. 1, in the retracted configuration;

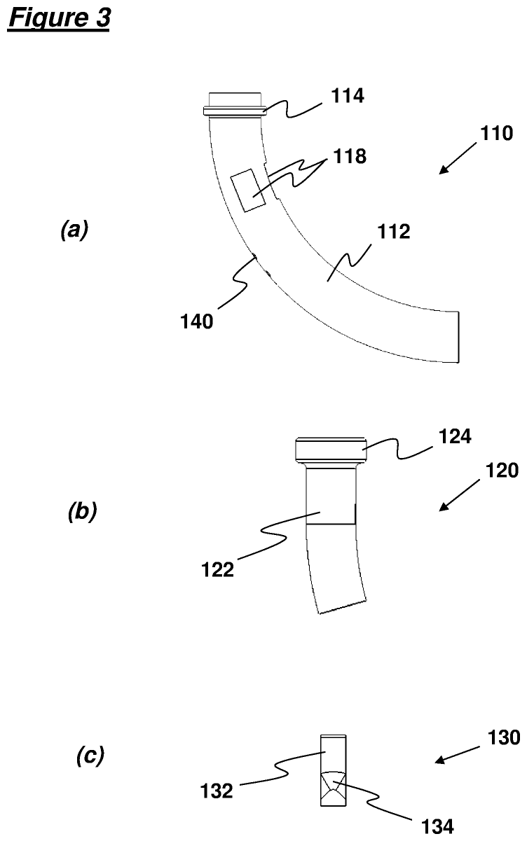

FIGS. 3a, 3b and 3c are side views of an outer cannula component, an inner cannula component and a retention member component, respectively, which each form part of the tracheostomy airway device of FIGS. 1 and 2;

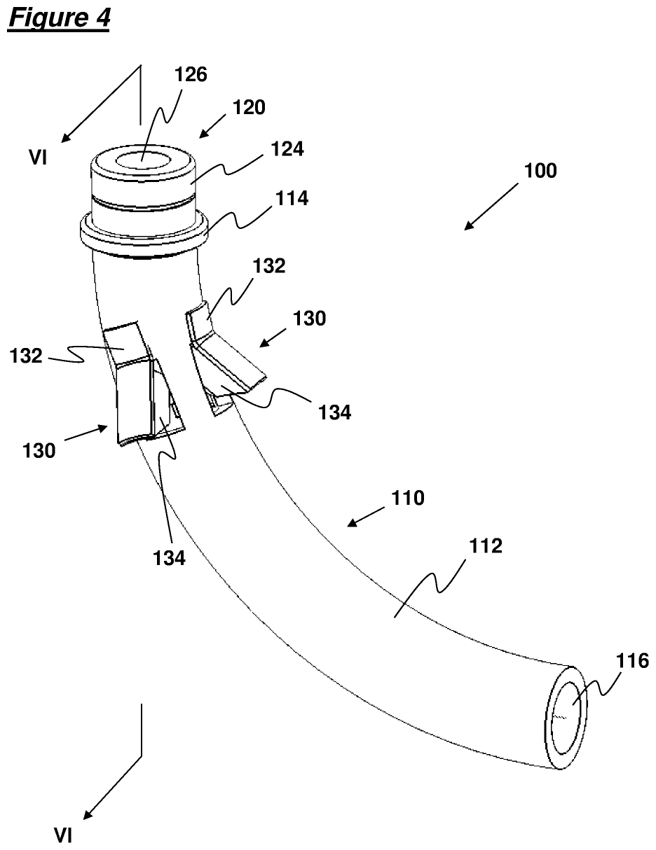

FIG. 4 is a third perspective view of the tracheostomy airway device of the previous figures, in an extended configuration;

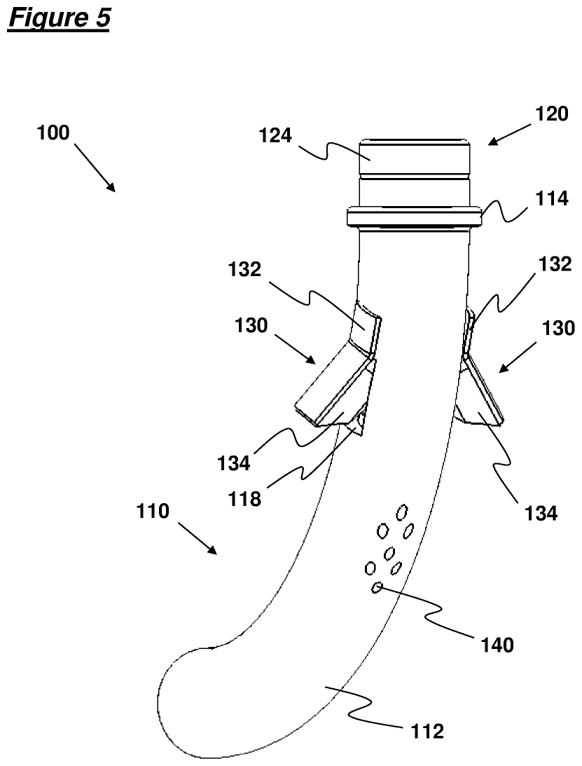

FIG. 5 is a fourth perspective view of the tracheostomy airway device of the previous figures, in an extended configuration;

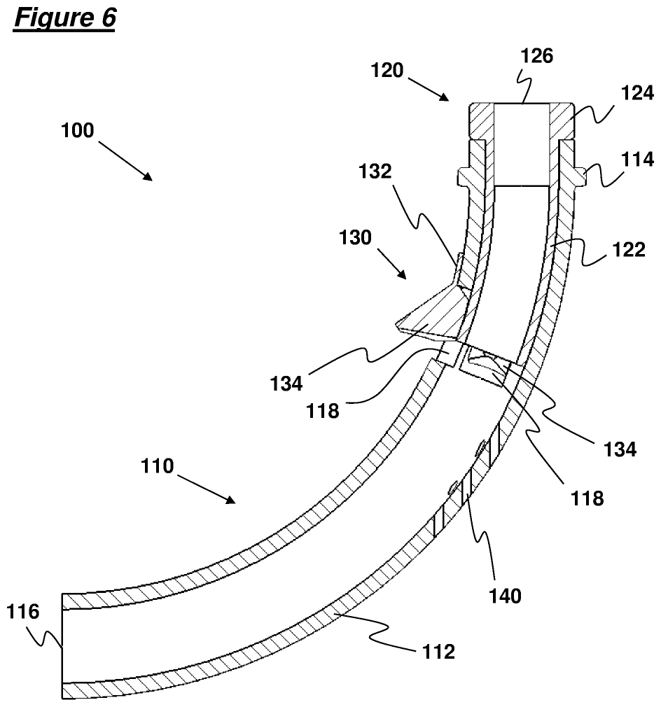

FIG. 6 is a cross-sectional view of the tracheostomy airway device of the previous figures, in an extended configuration, along line VI-VI in shown in FIG. 4; and

FIG. 7 is a schematic view of a conventional tracheostomy airway device inserted into the trachea of a patient.

A tracheostomy airway device according to the invention is shown in FIGS. 1 and 2, in a retracted configuration, and is generally designated 100. The tracheostomy airway device 100 comprises an outer cannula component 110, an inner cannula component 120 and three retention member components 130.

The outer cannula component 110, which is shown in isolation in FIG. 3a, is tubular, with a generally circular tubular wall 112. The tubular wall 112 has a short proximal portion that is generally cylindrical in shape, and a main portion that extends along an arcuate central axis, with the central axis having a substantially constant radius. The inner and outer diameters of the tubular wall 112 are uniform along the length of the outer cannula component 110, save for an abutment flange 114, which projects radially outwardly from the tubular wall 112 near to, but separately slightly from, a proximal end of the tubular wall 112. The abutment flange separates the generally cylindrical proximal portion of the tubular wall 112 and the main portion of the tubular wall 112 that extends along an arcuate central axis. The abutment flange 114 is annular in shape, with flat annular abutment surfaces, and a cylindrical side surface. The inner and outer surfaces of the tubular wall 112, save for the abutment flange 114, are smooth, and terminate at proximal and distal ends, which comprise flat annular end surfaces, orientated perpendicularly to the adjacent central axis of the tubular wall 12.

The outer cannula component 110 also comprises three rectangular openings 118 in the tubular wall 112, which are positioned approximately one-quarter of the length of the tubular wall 112 away from the proximal end. A first rectangular opening 118 has a central, longitudinal or major axis that is aligned with the plane of curvature of the arcuate central axis of the tubular wall 112, and is disposed on the radially-inner side of the arcuate central axis of the tubular wall 112. The second and third rectangular openings 118 are arranged at 90° to the first rectangular opening, and hence are disposed diametrically opposite each other.

As shown in FIG. 2, the outer cannula component 110 also comprises a group of seven circular openings in the tubular wall, this group being positioned approximately equidistant from the proximal and distal ends of the outer cannula component 110, and on the radially-outer side of the arcuate central axis of the tubular wall 112.

The inner cannula component 120, which is shown in isolation in FIG. 3b, is tubular, with a generally circular tubular wall 122. The tubular wall 122 has a proximal portion with an enlarged external diameter, and hence an increased wall thickness, which defines a head 124 of the inner cannula 120. The external diameter of the inner cannula head 124 is approximately equal to the external diameter of the tubular wall 112 of the outer cannula component 110. The tubular wall 122 also comprises a cylindrical intermediate portion, extending along a linear central axis, and a distal portion that extends along an arcuate central axis, with the central axis of the distal portion having a substantially constant radius. The inner and outer diameters of the tubular wall 122 are uniform along the length of the inner cannula component 120, save for inner cannula head 124, which projects radially outwardly from the tubular wall 122 at the proximal end of the tubular wall 122. The outer diameter of the intermediate and distal portions of the inner cannula component 120 is approximately equal to the internal diameter of the tubular wall 112 of the outer cannula component 110. The inner cannula head 124 is annular in shape, with flat annular abutment surfaces, and a cylindrical side surface. The inner and outer surfaces of the tubular wall 122, save for the inner cannula head 124, are smooth, and the tubular wall 122 terminates at proximal and distal ends, which comprise flat annular end surfaces, orientated perpendicularly to the adjacent central axis of the tubular wall 122.

The inner cannula component 120 is slidably received within the proximal end of the outer cannula component 110, and is insertable into the proximal end of the outer cannula component 110. In a retracted configuration of the tracheostomy airway device 100, as shown in FIGS. 1 and 2, the inner cannula component 120 is inserted into the proximal end of the outer cannula component 110, with the entire arcuate distal portion and approximately half of the linear intermediate portion of the tubular wall 122 of the inner cannula component 120 being accommodated within the tubular wall 112 of the outer cannula component 110. The remaining half of the linear intermediate portion of the tubular wall 122 of the inner cannula component 120, and the inner cannula head 124, thereby projecting from the proximal end of the outer cannula component 110, as shown in FIGS. 1 and 2.

The inner cannula component 120 is slidably engageable with the outer cannula component 110 along a continuum of positions until a fully engaged position, in the extended configuration of the tracheostomy airway device 100, in which the inner cannula head 124 of the inner cannula component 120 abuts the proximal end surface of the outer cannula component 110. This engagement and the extended configuration of the tracheostomy airway device 100 is discussed in more detail below, with reference to FIGS. 4 to 6.

The three retention member components 130, one of which is shown in isolation in FIG. 3c, are all identical in form. Each retention member component 130 comprises a rectangular, planar body 132, and a tapered projection 134 extending from a distal region of one of the two major surfaces of the rectangular, planar body 132. The tapered projection 134 is generally pyramidal in shape, with four generally triangular surfaces separated by linear edges, first and second surfaces being aligned along a central longitudinal axis of the rectangular, planar body 132, and third and fourth surfaces to each side of this central longitudinal axis.

Each retention member component 130 is bonded to a region of the exterior surface of the tubular wall 112 of the outer cannula component 110 on the proximal side of a corresponding rectangular opening 118, such that the rectangular, planar body 132 of the retention member component 130 overlies the corresponding rectangular opening 118, in the retracted configuration of the tracheostomy airway device 100. In this configuration, the tapered projection 134 of each retention member component 130 extends through the corresponding rectangular opening 118, such that the tapered projection 134 of each retention member component 130 projects into the interior of the outer cannula component 110, and in particular projects radially inwardly relative to the inner surface of the tubular wall 112 of the outer cannula component 110.

The retention member component 130 may be bonded to the exterior surface of the tubular wall 112 of the outer cannula component 110 with any suitably robust form of attachment, such as by welding, by adhesive or by co-moulding, for example.

Turning now to FIGS. 4 to 6, as discussed above, the inner cannula component 120 is slidably engageable with the outer cannula component 110 along a continuum of positions until a fully engaged position, in the extended configuration of the tracheostomy airway device 100, in which the inner cannula head 124 of the inner cannula component 120 abuts the proximal end surface of the outer cannula component 110. This extended configuration is shown in FIGS. 4 to 6.

As the inner cannula component 120 is slidably moved along the interior surface of the outer cannula component 110, along a continuum of positions, towards the fully engaged position, the distal end surface of the inner cannula component 120 comes into contact with, and abuts, the first surfaces of the tapered projections 134 of the three retention member components 130. Since the first surfaces of the tapered projections 134 of the three retention member components 130 are obliquely angled relative to the direction of movement of the inner cannula component 120, further movement of the inner cannula component 120 relative to the outer cannula component 110 causes the distal end of the inner cannula component 120 to urge the tapered projections 134 of the three retention member components 130, and the corresponding underling portions of the rectangular, planar bodies 132, outwardly relative to the outer cannula component 110. Each retention member component 130 deforms about a linear hinge, aligned along the proximal edge of the rectangular opening 118 of the outer cannula component 110, as the tapered projections 134 of the three retention member components 130, and the corresponding underling portions of the rectangular, planar bodies 132, are urged outwardly relative to the outer cannula component 110.

In the extended configuration of the tracheostomy airway device 100, in which the inner cannula head 124 of the inner cannula component 120 abuts the proximal end surface of the outer cannula component 110, the tubular wall 110 of the inner cannula component 120 lies alongside, and on the radially-inner side of, the first surfaces of the tapered projections 134 of the three retention member components 130. In this configuration, the three retention member components 130 at least partially project relative to the exterior surface of the outer cannula component 110, presenting an obliquely angled, planar surface towards the proximal end of the tracheostomy airway device 100.

Although not shown in the figures, the tracheostomy airway device 100 will typically also include a fastener component or so-called ‘neck flange’, bonded to the abutment flange 114 of the outer cannula component 120, which includes two generally planar wings that extend outwardly from the outer cannula component 110, and from which ribbon ties extend for securing the tracheostomy airway device 100 around the neck of a patient.

In use, an incision is made in the anterior aspect of the neck of a patient and in the trachea, thereby opening a direct airway into the trachea. The distal end of the tracheostomy airway device 100, in the retracted configuration (as shown in FIGS. 1-3), is then inserted through resulting stoma, or tracheostomy, until the abutment flange 114 is located externally adjacent to the exterior surface of the patient's neck, and the distal end of the outer cannula component 110 is located within the trachea of the patient, in the same manner as for a conventional tracheostomy airway device, as shown in FIG. 7. The inner cannula component 120 may, or may not, be present during insertion, and may be omitted where an obturator is used. Where a fastener component is provided, the ribbon ties are then passed around the neck of a patient, and tied, to secure the tracheostomy airway device 100 to the neck of the patient.

Once the tracheostomy airway device 100 has been inserted into the neck of a patient, as illustrated for a conventional tracheostomy airway device in FIG. 7, the three retention member components 130 are located within the trachea of the patient. The inner cannula component 120 is then slidably engaged with the outer cannula component 110 along a continuum of positions until a fully engaged position, in the extended configuration of the tracheostomy airway device 100, in which the inner cannula head 124 of the inner cannula component 120 abuts the proximal end surface of the outer cannula component 110, as shown in FIGS. 4-6. In this configuration, the three retention member components 130 at least partially project relative to the exterior surface of the outer cannula component 110, presenting an obliquely angled, planar surface towards the proximal end of the tracheostomy airway device 100 and the adjacent wall of the trachea. The three retention member components 130 would therefore impinge on the interior surface of the trachea of the patient in the event that the tracheostomy airway device 100 was subject to a removal force, such that the retention member components 130 would reduce the risk of the tracheostomy airway device 100 becoming dislodged or at least would reduce the risk of the tracheostomy airway device 100 becoming sufficiently dislodged to cause complications for the patient.

In order to remove the tracheostomy airway device 100, the inner cannula component 120 is slidably withdrawn, at least partially, from the outer cannula component 110 along a continuum of positions until the distal end of the inner cannula component 120 disengages from the tapered projections 134 of the three retention member components 130, such that the three retention member components 130 reform and withdraw into the rectangular openings 118 of the outer cannula component 110, until the three retention member components 130 no longer project outwardly relative to the outer cannula component 110, in the retracted configuration of the tracheostomy airway device 100, as shown in FIGS. 4-6. The tracheostomy airway device 100 is then withdrawn fully from the neck of the patient.

1. A tracheostomy airway device comprising an outer cannula having open proximal and distal ends, an inner actuation member insertable into the proximal end of the outer cannula, and one or more retention members, which are movable relative to the outer cannula between a retracted configuration in which the tracheostomy device is insertable into, and removable from, the trachea of a patient, and an extended configuration in which the one or more retention members impede removal of the tracheostomy airway device from the trachea, wherein the retention members are operably linked to the inner actuation member or the outer cannula, such that movement of the inner actuation member relative to the outer cannula actuates movement of the one or more retention members between the retracted configuration and the extended configuration.

2. A tracheostomy airway device as claimed in claim 1, wherein the inner actuation member has the form of an inner cannula.

3. A tracheostomy airway device as claimed in claim 1, wherein the inner actuation member may comprise a removable liner for facilitating cleaning of secretions that may accumulate, during use.

4-7. (canceled)

8. A tracheostomy airway device as claimed in claim 1, wherein the exterior shape of the inner actuation member substantially matches the interior shape of the outer cannula, at least those portions that are brought alongside each other on insertion of the inner actuation member into the outer cannula, such that the inner actuation member is received within the outer cannula with a close fit.

9.-10. (canceled)

11. A tracheostomy airway device as claimed in claim 1, wherein the inner actuation member comprises an engagement portion that is either adapted to come into engagement with the one or more retention members, or where the inner actuation member is fixed to the one or more retention members, the engagement portion is adapted to come into engagement with the outer cannula or other engagement member fixed to the outer cannula.

12. A tracheostomy airway device as claimed in claim 1, wherein the inner actuation member is fixable relative to the outer cannula, following insertion and movement to the extended configuration, in order to provide a configuration suitable for long-term use of the device by the patient.

13. (canceled)

14. A tracheostomy airway device as claimed in claim 1, wherein the one or more retention members comprise an engagement portion that is engaged by a corresponding engagement portion of the inner actuation member, during insertion of the inner actuation member into the outer cannula, in use.

15. (canceled)

16. A tracheostomy airway device as claimed in claim 14, wherein the one or more retention members are arranged for movement relative to the outer cannula as the inner actuation member is inserted into the outer cannula, and the engagement portion of the inner actuation member moves the engagement portion of the one or more retention members.

17. A tracheostomy airway device as claimed in claim 1, wherein the one or more retention members are mounted relative to the inner actuation member.

18. A tracheostomy airway device as claimed in claim 17, wherein the one or more retention members comprise an engagement portion that is engaged by a corresponding engagement portion of the outer cannula, during insertion of the inner actuation member into the outer cannula, in use.

19. A tracheostomy airway device as claimed in claim 18, wherein the one or more retention members are arranged for movement relative to the outer cannula as the inner actuation member is inserted into the outer cannula, and the engagement portion of the outer cannula moves the engagement portion of the one or more retention members.

20-22. (canceled)

23. A tracheostomy airway device as claimed in claim 1, wherein the one or more retention members comprise a retaining portion, which projects outwardly relative to the exterior surface of the outer cannula in the extended configuration and is arranged to impinge on the interior surface of the trachea of a patient, in the extended configuration of the device, in the event that a removal or dislodgement force and movement is applied to the device.

24.-25. (canceled)

26. A tracheostomy airway device as claimed in claim 1, wherein the one or more retention members are mounted to the outer cannula or the inner actuation member by means of a fixed portion of the one or more retention members, which does not move relative to the component to which it is fixed, and the one or more retention members being deformable.

27. A tracheostomy airway device as claimed in claim 26, wherein the portion of the one or more retention members that is deformable relative to the fixed portion may comprise a retaining portion and/or an engagement portion of the one or more retention members.

28.-29. (canceled)

30. A tracheostomy airway device as claimed in claim 1, wherein the one or more retention members are subject to a biasing force, which acts to urge the one or more retention members to a rest position which corresponds to the position of the one or more retention members in a retracted configuration, when the inner actuation member is withdrawn, e.g. at least partially or fully, from the outer cannula.

31-35. (canceled)

36. A tracheostomy airway device as claimed in claim 23, wherein a retaining portion of the one or more retention members comprises an abutment surface arranged to impinge on an interior surface of the trachea of a patient, in the event of a withdrawal or dislodgement force or movement.

37. A tracheostomy airway device as claimed in claim 36, wherein the abutment surface is orientated obliquely relative to the axis along which the corresponding portion of the outer cannula extends, and relative to the interior surface of the trachea.

38. (canceled)

39. A tracheostomy airway device as claimed in claim 36, wherein a retaining portion of the one or more retention members is movable between a position in the retracted configuration where the abutment surface is substantially aligned with the exterior surface of the outer cannula, and a position in the extended configuration in which the retaining portion and/or the abutment surface projects outwardly relative to the exterior surface of the outer cannula.

40.-41. (canceled)

42. A method of inserting a tracheostomy airway device, such as the tracheostomy device defined in claim 1, comprising:
(a) providing the tracheostomy device in a retracted configuration in which the tracheostomy device is insertable into the trachea of a patient;
(b) inserting an outer cannula of the tracheostomy device into the trachea of a patient;
(c) inserting an inner actuation member of the tracheostomy device into a proximal end of the outer cannula; and
(d) moving the inner actuation member relative to the outer cannula, thereby actuating movement of one or more retention members of the tracheostomy device into an extended configuration in which the one or more retention members impede removal of the tracheostomy airway device from the trachea.

43. A method as claimed in-claim 42, wherein the method includes a further step, for decannulation, of moving the inner actuation member relative to the outer cannula, thereby actuating movement of one or more retention members of the tracheostomy device into the retracted configuration in which the tracheostomy device is removable from the trachea of a patient.
